(19) United States
(12) Patent Application Publication
(10) Pub. No.: US 2007/0107263 A1
(43) Pub. Date: May 17, 2007

(54) Heel insert

(76) Inventor: Douglas E. Lopez, Pueblo, CO (US)

Correspondence Address: BARBER LEGAL, P.O. BOX 16220, GOLDEN, CO, 80402-6004, US

(21) Appl. No.: 11/271,631

(22) Filed: Nov. 11, 2005

(51) Int. Cl. A43B 19/00; A61F 5/14

(52) U.S. Cl. 36/71; 36/140; 36/144

(57) ABSTRACT

The present invention teaches that an improved shoe insert may be made in a smaller size and of a material which is firmer and more resilient, yet flexible than in prior art devices. An advantageous trapezoidal planform is also taught, in which the rear edge is shorter than the parallel front edge. The device may have a rear edge having a thickness of a few millimeters (up to ⅛ inch) while the thickness of the device decreases regularly to the front edge, at which point the thickness reaches zero. The size of the device may advantageously be in the range from 1 inch in rear length to 1.5 inches in rear length, 1.25 inch in front length to 1.75 inch in front length, and from 1 inch in width (defined as the distance separating the parallel rear and front edges) to 1.25 inches in width.

### STATEMENT REGARDING FEDERALLY FUNDED RESEARCH

This invention was not made under contract with an agency of the US Government, nor by any agency of the US Government.

### FIELD OF THE INVENTION

This invention relates generally to shoes and specifically to retrofit-able heel inserts for shoes.

### BACKGROUND OF THE INVENTION

Variation in individual's shape and size of feet mean that different individuals find that different shapes of shoe support provide comfort. As a result, different shoes provide different levels of comfort for different people. However, this by itself does not provide comfort at all times.

For example, most individuals find that their feet are just slightly different sizes or shapes. An individual may have a leg discrepancy, and some individuals have foot pronation or supination or these conditions may just occur in a sole, heel or foot. Other individuals find that no pair of shoes really provides real comfort for personal reasons of choice, foot shape and so on.

One known solution is the insole insert, an extremely soft “foot shaped” layer of material for insertion (retrofit) into a shoe.

However, traditional insole shoe inserts present problems to most users. In particular the insole insert takes up space covering a substantial portion of the planform of the shoe. This results in the insole insert taking up excessive space inside the shoe, often meaning that users cannot wear them due to discomfort of toes or some other part of the foot.

In addition, most insole inserts are composed of an extremely soft material which compacts easily down to some minimal thickness, thus robbing the user of most of the theoretical benefit of the device.

Yet another problem with the insole insert is that it is essentially “foot shaped”, that is it is geared towards users who have some “whole foot” discomfort, as opposed to those who find that they need particular support at a particular point of the foot.

Another known solution is the arch support, a device whose shape is defined by the space between the sole of the shoe and the arches of some individuals. However, this particular solution is specialized and suits only those individuals who have this exact problem and have the exact shape of arch for which the support is designed.

It would be advantageous to provide a device offering a degree of very firm support, able to sustain the actual weight of the user.

It would further be advantageous to provide a device small enough to offer support to only one portion of the user's foot, without taking up space in other parts of the shoe.

It would further be advantageous to provide a device of a shape able to fit easily into a variety of locations in a shoe.

It would further be advantageous to provide a device firm enough to support a user's weight at the heel, yet resilient enough to provide comfort to the user.

### SUMMARY OF THE INVENTION

General Summary

The present invention teaches that an improved shoe insert may be made in a smaller size and of a material which is firmer yet flexible and more resilient than in prior art devices. An advantageous trapezoidal planform is also taught, in which the rear edge is shorter than the parallel front edge. The device may have a rear edge having a thickness of a few millimeters (up to ⅛ inch (0.375 inch)) while the thickness of the device decreases regularly to the front edge, at which point the thickness reaches zero. The size of the device may advantageously be in the range from 1 inch in rear length to 1.5 inches in rear length, 1.25 inch in front length to 1.75 inch in front length, and from 1 inch in width (defined as the distance separating the parallel rear and front edges) to 1¼ inches in width.

The device may also be made in a rectangular embodiment of approximately the same dimensions, although the trapezoidal embodiment allows more support and cushion underneath the foot.

One material which has been determined to have the desirable hard yet highly resilient properties is rubber, specifically NEOPRENE™ brand rubber, a blend of styrene butadiene rubber (SBR), neoprene (CR) and nitrile rubber (NBR). It is the most flexible of the rubber family. Shore A Durometer 30 is presently used and test to show that the tensile strength is 1000 PSI at 1/32 inch to 1 inch gauge thickness and will support from 125 to 175 pounds of weight repeatedly applied and also for long durations of time before any compression sets in.

NEOPRENE™ as used in the device may be extruded.

Summary in Reference to Claims

It is therefore another aspect, advantage, objective and embodiment of the present invention to provide a shoe insert comprising:
a body of a firm resilient material;
the body having a bilaterally symmetrical trapezoidal planform having four sides: a back, a front, and two sides, the back side parallel to and shorter than the front side, the body having rounded corners;
the body having in cross section a first thickness at the back side, the thickness of the body decreasing regularly to a second thickness the front side;
the body having a bottom surface and a top surface;
an adhesive layer disposed upon the bottom surface of the body;
a backing layer disposed upon the adhesive layer.

It is therefore another aspect, advantage, objective and embodiment of the present invention to provide a shoe insert wherein the thickness at the front side is zero millimeters.

It is therefore another aspect, advantage, objective and embodiment of the present invention to provide a shoe insert wherein the thickness at the back side is 0.125 inch.

It is therefore another aspect, advantage, objective and embodiment of the present invention to provide a shoe insert wherein the firm resilient material comprises rubber.

It is therefore another aspect, advantage, objective and embodiment of the present invention to provide a shoe insert wherein the rubber comprises NEOPRENE brand rubber.

It is therefore another aspect, advantage, objective and embodiment of the present invention to provide a shoe insert wherein the length of the short edge is 1.25 inch.

It is therefore another aspect, advantage, objective and embodiment of the present invention to provide a shoe insert wherein the length of the long edge is 1.5 inch.

It is therefore another aspect, advantage, objective and embodiment of the present invention to provide a shoe insert wherein the distance between the short edge and the long edge is 1.125 inch.

It is therefore another aspect, advantage, objective and embodiment of the present invention to provide a shoe insert wherein the adhesive further comprises any high bond semi-permanent adhesive.

It is therefore another aspect, advantage, objective and embodiment of the present invention to provide a shoe insert comprising:
a body of a firm resilient material;
the body having a planform having four sides: a back, a front, and two sides, the back side parallel to and the same length as the front side, the body having rounded corners;
the body having in cross section a first thickness at the back side, the thickness of the body decreasing regularly to a second thickness the front side;
the body having a bottom surface and a top surface;
an adhesive layer disposed upon the bottom surface of the body which may be very high bond (VHB) semi-permanent adhesive, known for its strength and durability in extreme applications,
a backing layer disposed upon the adhesive layer.

### BRIEF DESCRIPTION OF THE DRAWINGS

FIG. 1 is a planform top view of a first embodiment of the invention.

FIG. 2 is a cross-sectional side view of the first embodiment of the invention.

FIG. 3 is a left side view of the first embodiment of the invention.

FIG. 4 is a right side view of the first embodiment of the invention.

FIG. 5 is a bottom view of the first embodiment of the invention.

FIG. 6 is a rear view of the first embodiment of the invention.

FIG. 7 is a front view of the first embodiment of the invention.

FIG. 8 is a planform top view of a second embodiment of the invention.

FIG. 9 is a cross-sectional side view of the first embodiment of the invention.

FIG. 10 is a left side view of the first embodiment of the invention.

FIG. 11 is a right side view of the first embodiment of the invention.

FIG. 12 is a bottom view of the first embodiment of the invention.

FIG. 13 is a rear view of the first embodiment of the invention.

FIG. 14 is a front view of the first embodiment of the invention.

FIG. 15 is a partial top view of a shoe with the invention in use in a first manner.

FIG. 16 is a partial top view of a shoe with the invention in use in a second manner.

FIG. 17 is a partial top view of a shoe with the invention in use in a third manner.

### INDEX TO REFERENCE NUMERALS

Body 100
Top surface 102
Rear side 104
Front side 106
Right side 108
Left side 110
Bottom surface 112
Adhesive layer 114
Backing layer 116
Rounded corner 120, 122, 124, 126
Body 200
Top surface 202
Rear side 204
Front side 206
Right side 208
Left side 210
Bottom surface 212
Adhesive layer 214
Backing layer 216
Rounded corner 220, 222, 224, 226
Shoe 500
Shoe upper 502
Heel insert 504
Shoe interior 506
Shoe 600
Heel insert 604
Shoe 700
Heel insert 704

### DETAILED DESCRIPTION

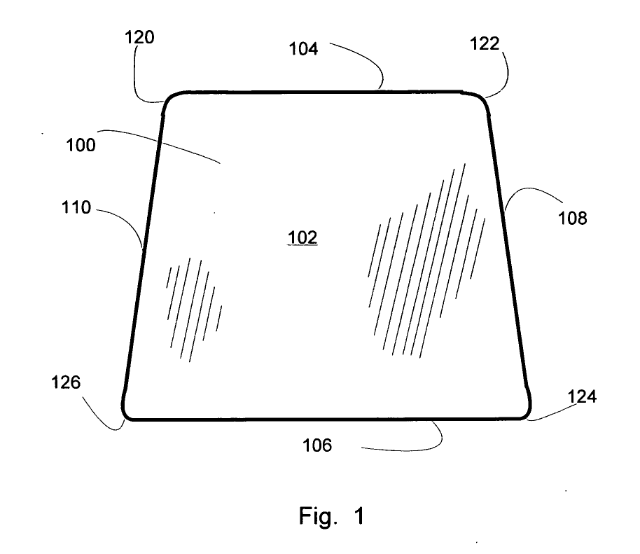

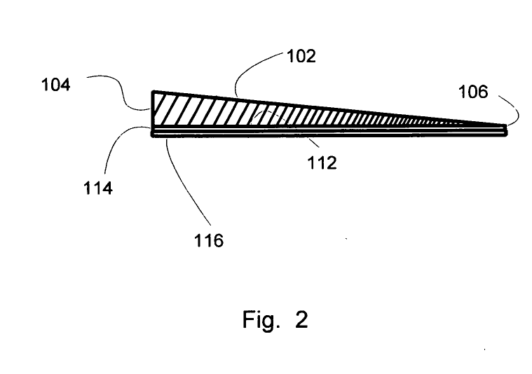

FIG. 1 is a planform top view of a first embodiment of the invention. FIG. 2 is a cross-sectional side view of the first embodiment of the invention. These diagrams of the first embodiment of the invention show the presently preferred embodiment of the invention, which is also the best mode now contemplated for carrying out the invention.

Body 100 is a trapezoidal shape in planform (FIG. 1), an acute triangle viewed from the side (FIG. 2). Top surface 102 is thus trapezoidal and defined by four sides: rear side 104 and front side 106, right side 108, left side 110.

Rear side 104 and front side 106 are parallel to each other, while right side 108 and left side 110 may be symmetrical (in the preferred embodiment) or asymmetrical in angle. In the best mode now contemplated, rear side 104 is shorter than front side 106 (front side 106 is longer than rear side 104).

Rounded corners 120, 122, 124, 126 at the junctions of the sides provide a firmer grip and increased comfort. Without wishing to be bound by any theories, is it believed that sharp corners may be sensed by the bottom of the foot, leading to potential discomfort, and that sharp corners are more likely to lose adhesion, begin to project upwards, and thus turn into a “snag” waiting to get caught on a user's sock and then cause the entire device to lift away from the sole of the shoe.

This may also be placed under a removable insole a shoe.

The trapezoidal configuration offers a number of benefits. One benefit is that the narrowing of the rear side allows the device to be used in narrower potions of shoes. In particular, the rear end of the shoe narrows considerably, and a narrower rear edge allows the insert of the invention to sit closer to the rear of the shoe.

Bottom surface 112 (FIG. 2) has an adhesive layer 114 and backing layer 116. Adhesive layer 114 may be an adhesive which may be very high bond (VHB) semi-permanent adhesive, known for its strength and durability in extreme applications. Backing layer 116 may be used to prevent the adhesive from becoming dirty prior to the desire use.

In one preferred embodiment, the adhesive is a glue having a high degree of “tack” (i.e. the ability to be lifted up and set in a new location and still stick) and may be semi-permanent even under extreme conditions.

Cross sectional FIG. 2 also shows the “wedge” shape of the device: an acute triangle narrowing to zero thickness at the front side. The wedge shape produces torsion control which keeps the heel from moving from side to side. The first thickness at the rear side or rear edge 104 may advantageously be roughly 3 millimeters (about ⅛ inch), then the thickness in a regular manner (at a constant rate) decreases until the thickness is zero at the front side or front edge 106. Obviously, the point at which thickness reaches zero may be said to define the front side 106.

Body 100 is itself a hard or firm resilient material. While most prior art devices are soft pliable fabrics, the invention is not soft and pliable. On the contrary, the body 100 of the invention is firm and resilient yet flexible. In the preferred embodiment, the firm resilient material comprises rubber, such as NEOPRENE™ brand rubber. This is a blend of styrene butadiene rubber (SBR), neoprene (CR) and nitrile rubber (NBR). It is the most flexible of the rubber family. Shore A Durometer 30 is presently used and tested to show that the tensile strength is 1000 PSI at 1/32 inch to 1 inch gauge thickness and will support from 125 to 175 pounds of weight repeatedly applied and also for long durations of time before any compression sets in.

Without wishing to be bound by any theory, it is believed that the reason a firm resilient body is superior to a soft and pliable body is that the device is intended to provide actual support of the weight of the user. That is, most prior art devices simply sit flat under the full area of the sole of the foot, or else are arch support devices which project upwards into the area under the arch of the foot. The invention provides actual support, for example under the heel of the foot, and can be conveniently placed where needed, for example so that the entire weight of the user will rest for a split second during each step the person takes.

Thus the shoe insert should comprise a body of a firm resilient material, the body bilaterally symmetrical and trapezoidal in planform with round corners and with the back side parallel to and shorter than the front side.

In cross section, the body has a first thickness at the back side and the thickness of the body decreasing regularly to a second thickness the front side, which is preferably zero thickness.

The body bottom surface or top surface may have an adhesive layer disposed thereon with a backing layer disposed upon the adhesive layer.

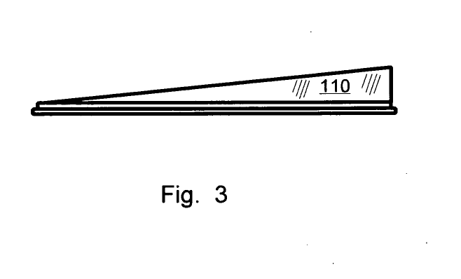

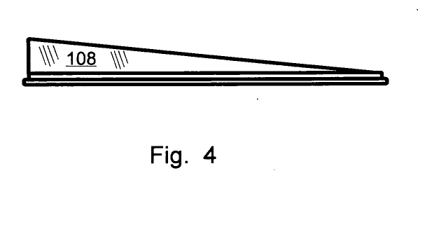

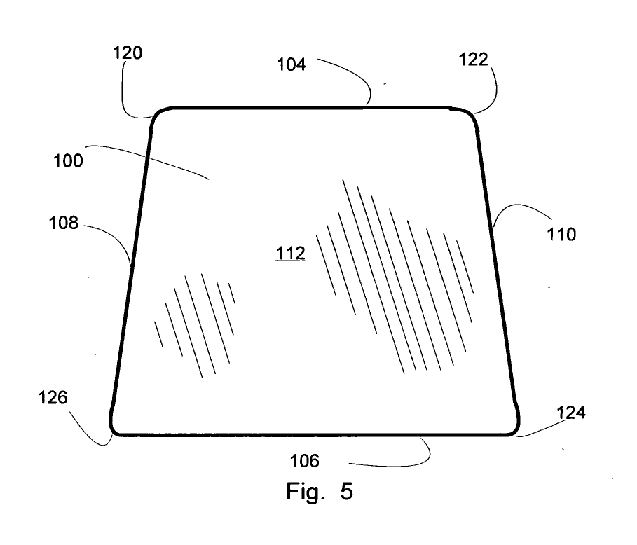

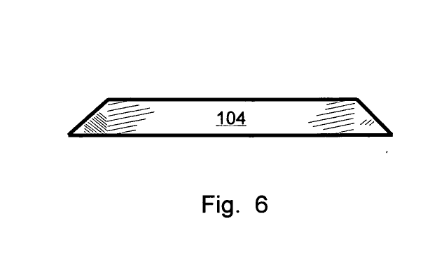

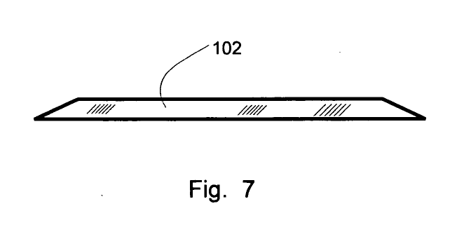

FIG. 3 is a left side view of the first embodiment of the invention. FIG. 4 is a right side view of the first embodiment of the invention. FIG. 5 is a bottom view of the first embodiment of the invention. FIG. 6 is a rear view of the first embodiment of the invention. FIG. 7 is a front view of the first embodiment of the invention. Sides 110, 108, and bottom and top surfaces 112 and 102 may be seen in these diagrams.

A back side thickness of 0.125 inch, (about 3 millimeters) has been tested and proven beneficial.

The size of the device may advantageously be in the range from 1 inch in rear length to 1.5 inches in rear length (25-40 millimeters), 1.25 inch in front length to 1.75 inch in front length (32-45 millimeters), and from 1 inch in width (defined as the distance separating the parallel rear and front edges) to 1.25 inches in width (25-30 millimeters).

A length of the short edge of 1.25 inch is presently most preferred, and a length of the long edge at 1.5 inch is presently most preferred. A distance between the short edge and the long edge of 1.125 inch is presently most preferred.

The trapezoidal shape provides is better than a rectangular shape for narrower spaces and provides more product underfoot, thus the trapezoidal shape is the presently preferred choice.

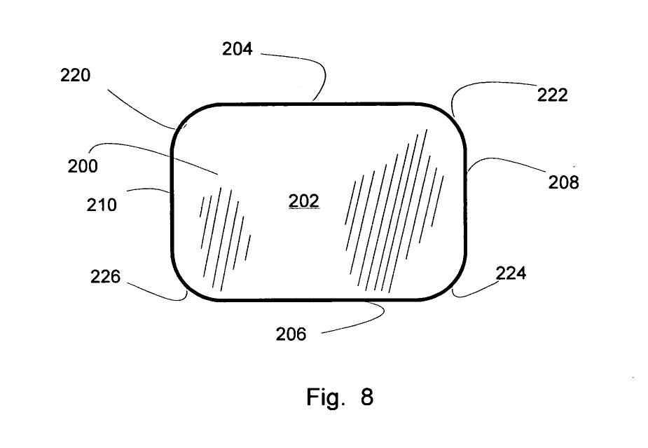

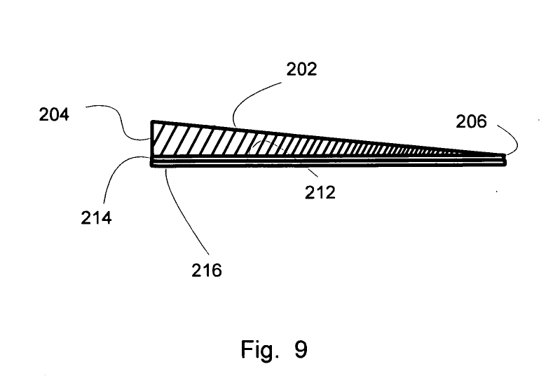

FIG. 8 is a planform top view of a second embodiment of the invention. FIG. 9 is a cross-sectional side view of the first embodiment of the invention. Body 200 is of a rectangular planform, rather than trapezoidal. Top surface 202, rear side 204, front side 206, right side 208, left side 210, and rounded corners 220, 222, 224, 226 are otherwise much as in the embodiment of FIGS. 1 through 7.

Thus the device may instead be a body of a firm resilient material having a rectangular planform having four sides: a back, a front, and two sides, the back side parallel to and the same length as the front side, and of course the sides also parallel and the same lengths, with rounded corners and a first thickness at the back side which decreases regularly to a second thickness the front side.

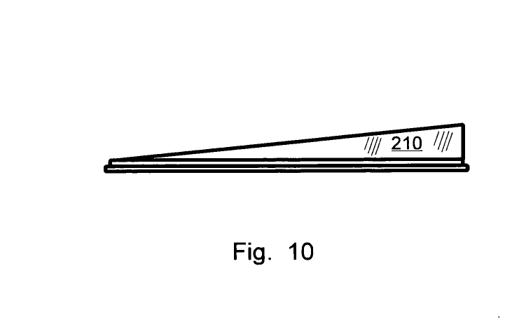

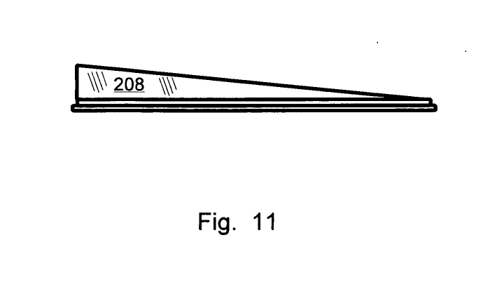

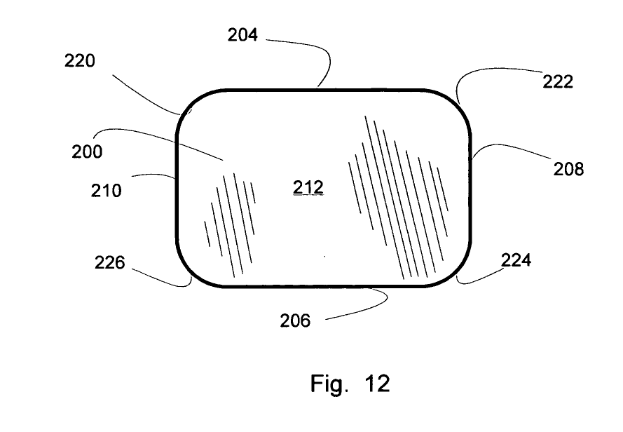

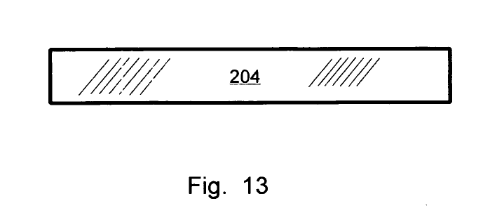

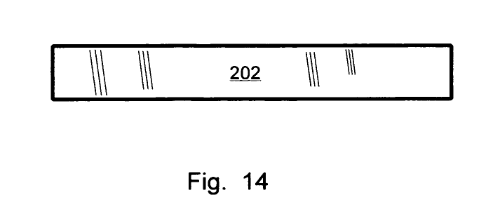

The bottom surface 212 and top surface 202 may then be much as disclosed: an adhesive layer 214 disposed upon the bottom surface with a backing layer 216 disposed upon the adhesive layer. FIG. 10 is a left side 210 view of the first embodiment of the invention. FIG. 11 is a right side 208 view of the first embodiment of the invention. FIG. 12 is a bottom view of the first embodiment of the invention. FIG. 13 is a rear view of the first embodiment of the invention. FIG. 14 is a front view of the first embodiment of the invention.

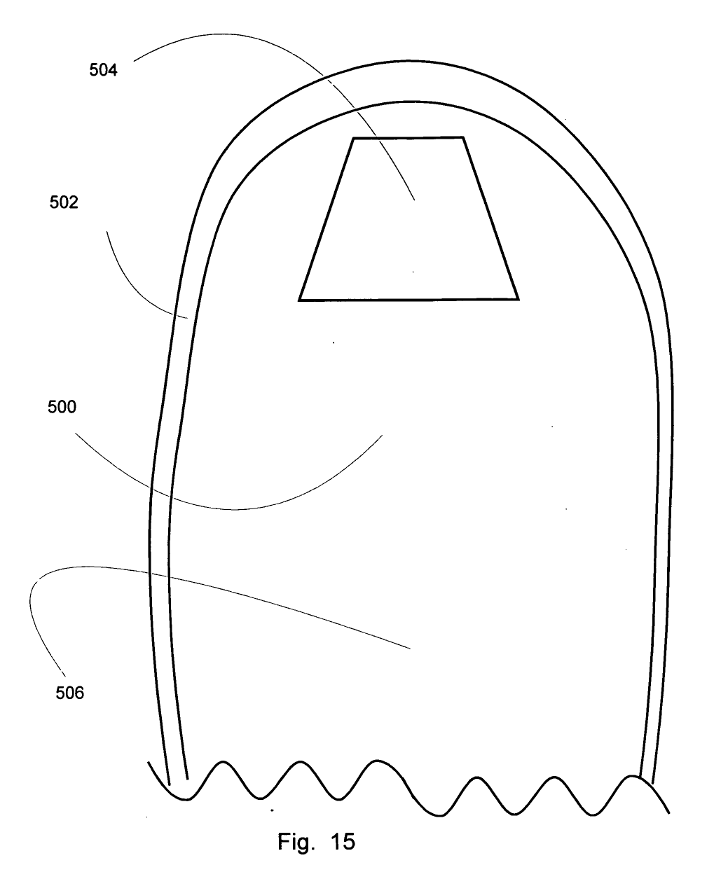

FIG. 15 is a partial top view of a shoe with the invention in use in a first manner. Shoe 500 has shoe upper 502 and shoe interior 506. Within shoe interior 506, heel insert 504 is placed so that it is facing with the sloped upper surface facing towards the front of the shoe, to provide additional heel support.

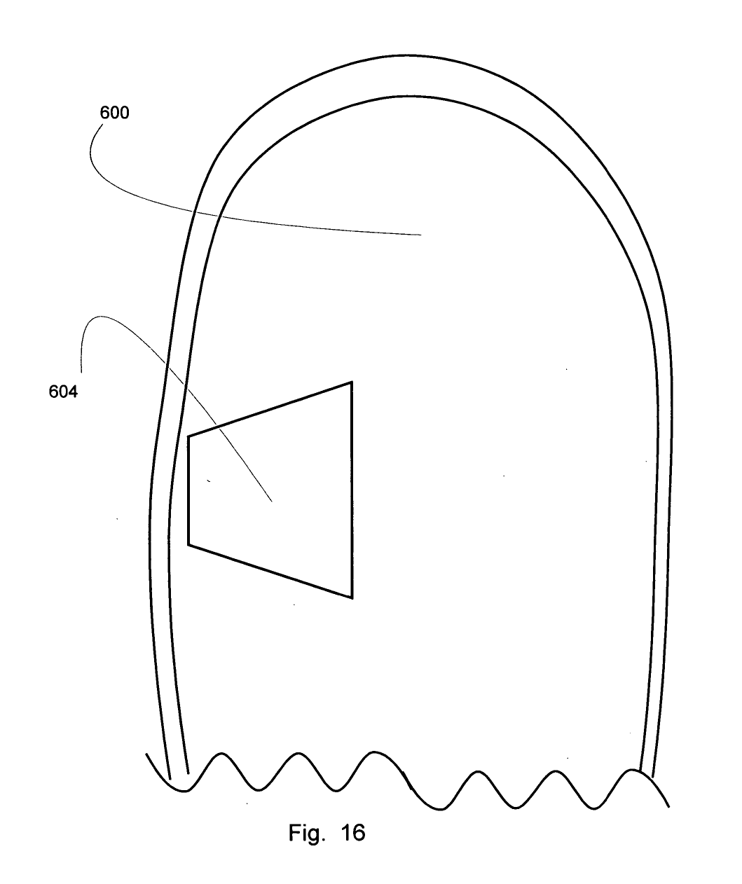

FIG. 16 is a partial top view of a shoe with the invention in use in a second manner. Shoe 600 has heel insert 604 placed facing inwards towards the centerline of the user's body so as to provide additional support to one with a pronate condition. This support will aid in straightening the ankle to a more vertical orientation.

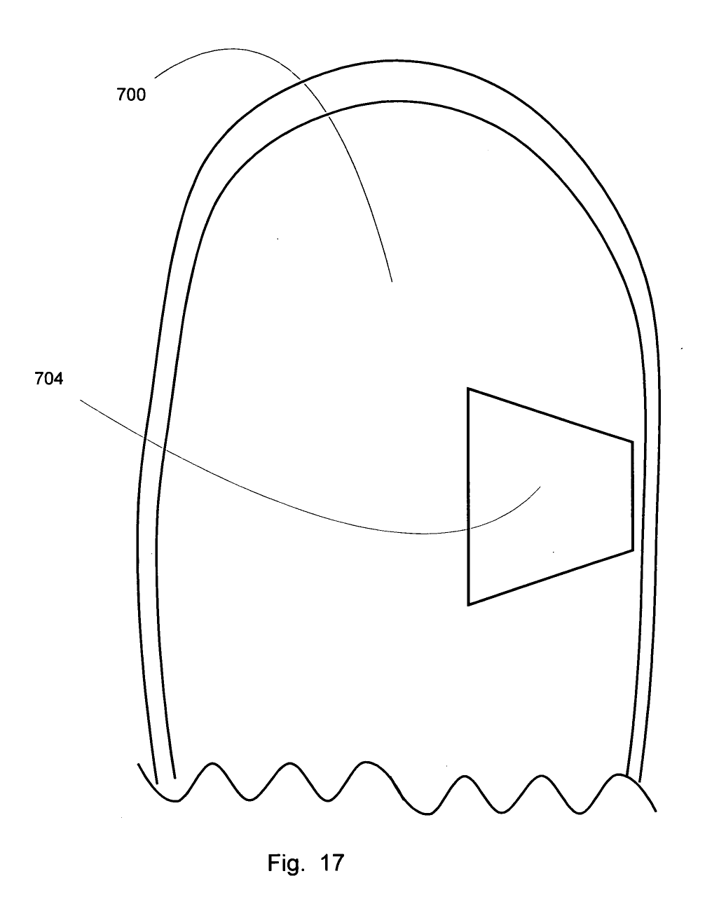

FIG. 17 is a partial top view of a shoe with the invention in use in a third manner. Shoe 700 has heel insert 704 facing outwards from the centerline of the user's body so as to provide additional support to one with a supination condition of the foot and ankle.

The disclosure is provided to allow practice of the invention by those skilled in the art without undue experimentation, including the best mode presently contemplated and the presently preferred embodiment. Nothing in this disclosure is to be taken to limit the scope of the invention, which is susceptible to various equivalents without departing from the scope and spirit of the invention. The scope of the invention is to be understood from the claims accompanying this disclosure.

I claim:

1. A shoe insert comprising:
a body of a firm, flexible and resilient material;
the body having a bilaterally symmetrical trapezoidal planform having four sides: a back, a front, and two sides, the back side parallel to and shorter than the front side, the body having rounded corners;
the body having in cross section a first thickness at the back side, the thickness of the body decreasing regularly to a second thickness the front side;
the body having a bottom surface and a top surface;
an adhesive layer disposed upon the bottom surface of the body;
a backing layer disposed upon the adhesive layer.

2. The shoe insert of claim 1, wherein the thickness at the front side is zero millimeters.

3. The shoe insert of claim 1, wherein the thickness at the back side is 0.125 inch.

4. The shoe insert of claim 1, wherein the firm flexible resilient material comprises rubber.

5. The shoe insert of claim 4, wherein the rubber comprises NEOPRENE rubber.

6. The shoe insert of claim 1, wherein the length of the short edge is 1.25 inch.

7. The shoe insert of claim 1, wherein the length of the long edge is 1.5 inch.

8. The shoe insert of claim 1, wherein the distance between the short edge and the long edge is 1.125 inch.

9. The shoe insert of claim 1, wherein the adhesive further comprises any high bond semi-permanent adhesive.

10. A shoe insert comprising:
a body of a firm flexible resilient material;
the body having a planform having four sides: a back, a front, and two sides, the back side parallel to and the same length as the front side, the body having rounded corners;
the body having in cross section a first thickness at the back side, the thickness of the body decreasing regularly to a second thickness the front side;
the body having a bottom surface and a top surface;
an adhesive layer disposed upon the bottom surface of the body;
a backing layer disposed upon the adhesive layer.
